(19) United States
(12) Patent Application Publication
(10) Pub. No.: US 2026/0248665 A1
(43) Pub. Date: Aug. 27, 2026

(54) PATIENT SUPPORT, WHEELCHAIR COMPRISING A PATIENT SUPPORT AND METHOD FOR THE AUTOMATED CALIBRATION OF A PATIENT SUPPORT

(71) Applicant: RELiYOO AG, Kienberg (CH)

(72) Inventors: Jürgen SCHONHARDT, Rheinfelden (DE); Uwe SCHONHARDT, Kienberg (CH); Nick SCHÄUBLE, Weil am Rhein (DE); Marcel KALBERER, Winterthur (CH)

(73) Assignee: RELiYOO AG, Kienberg (CH)

(21) Appl. No.: 19/135,625

(22) PCT Filed: Dec. 8, 2022
(86) PCT No.: PCT/EP2022/085027

§ 371 Date: Jun. 4, 2025

(51) Int. Cl. A61G 5/10

(52) U.S. Cl. CPC A61G 5/1045; A61G 5/1091; A61G 2203/10; A61G 2203/34; A61G 2203/40

(57) ABSTRACT

The invention relates to a patient support (101), preferably a seat cushion, comprising a support area (1) with a base (2), a plurality of fluid cells (3) arranged on a seat side of the base (2), a pump (4), and a control unit (5), wherein one or more fluid cells (3) each define a pressure zone (6) and each pressure zone (6) is connected to the pump (4) by at least one zone valve (7), wherein each zone valve (7) can be actuated by the control unit (5), so that a pressure in each pressure zone (6) can be set by the control unit (5), characterized in that the patient support (101) comprises at least one pressure sensor (8) for measuring the pressure in at least one pressure zone (6), preferably at least one pressure sensor (8) per pressure zone (6).

The invention relates to a patient support, a wheelchair comprising a patient support and a method for automated calibration of a patient support.

Pressure ulcers are tissue damage caused by prolonged pressure and shear stress. Such pressure typically occurs in immobile patients, e.g. those who are bedridden or wheelchair-bound

In the state of the art, patient supports are known that consist of a number of fluid cushions. These can be subjected to a specific pressure so that the patient positioned on the fluid cushion is not exposed to a constant pressure load despite immobility.

EP 2 8924 89 A1 discloses a uniformly inflatable mattress with a pressure sensor to support patients and prevent bedsores.

EP 1 643 882 A1 discloses a cellular cushion and method of manufacturing a cellular cushion which increases the surface area for supporting seated persons so that blood circulation through the tissue can be ensured.

EP 2 731 567 A2 relates to a patient/invalid transportation device that proposes pressure distribution of a patient by a plurality of inflatable cushions.

However, the known patient supports are not flexible and cannot be easily customized to the patient's tissue, so that certain areas can be particularly relieved.

The state of the art lacks a patient support that enables simple and intuitive operation and can be used flexibly to prevent damage to a patient's tissue, in particular due to a lack of blood flow to the tissue.

In addition, the state of the art lacks a patient support that prevents the patient from being positioned on one side. The state of the art also lacks an adaptable patient support that adapts to the orientation and positioning of a patient on the patient support and/or to the individual needs of the patient.

It is the object of the present invention to overcome these and other disadvantages of the prior art. In particular, the invention is intended to provide a simple patient support for relieving the pressure on vulnerable areas of a patient. In addition, it is intended to provide an automated patient support, in particular a patient support which allows separate relief of particularly endangered areas of a patient without impairing seating stability.

The task is solved by a patient support, a wheelchair and a method as defined in the independent claims. Further embodiments result from the dependent claims.

The patient support according to the invention comprises a support area with a base, a plurality of fluid cells, a pump and a control unit. Preferably, the patient support is a seat cushion, for example a cushion. The plurality of fluid cells are arranged on a seat side of the patient support. One or more fluid cells each define a pressure zone. Each pressure zone is connected to the pump by at least one zone valve. Each zone valve can be actuated independently by the control unit, so that a pressure in each pressure zone can be adjusted by the control unit. The patient support comprises at least one pressure sensor for measuring the pressure in at least one pressure zone. Preferably, at least one pressure sensor is arranged per pressure zone.

The terminology “protect/relieve” in this context means that areas are subjected to a lower overall pressure and/or lower punctual pressure of an area of the patient. This can be achieved, for example, by distributing the weight force over a larger area and/or exposing other areas to more pressure in order to compensate for the weight force acting on the area to be protected/relieved.

The terminology “support” in this context means that areas of the patient are supported evenly and over as large an area as possible so that stable positioning is possible.

The patient support can also include an energy storage device, e.g. an accumulator, for the power supply. This has the advantage that the patient support can be used on the move and is not dependent on a local power supply.

Alternatively, the pressure sensor can be arranged in the pump, in a fluid cell, a feed to the pressure zones or a feed to the fluid cells of the pump.

The pressure sensor can be fluid-connected or fluid-connectable to several pressure zones, preferably via a common supply of the pressure zones. The pressure zones can preferably be selectively connected to the pressure sensor, for example via the valves, in particular zone valves.

This means that exactly one pressure sensor, in particular exactly one pressure sensor, can be fitted to measure several pressure zones depending on the opening states of the valves, in particular zone valves. The pressure of different pressure zones can be measured independently of each other if, for example, only one zone valve is open.

The pressure sensor can be arranged in the supply or pressure zone, preferably the fluid cell, in particular adjacent to the inlet and/or outlet valve, so that the electrical setup is simplified. In particular, the pressure sensor can be arranged in the direction of flow of the gas during pressurization, after the zone valve, after the inlet valve or before the outlet valve.

One or more pressure zones can comprise at least two fluid cells that are fluid-connected to each other. This allows for a more uncomplicated design of the patient support, as fewer valves and/or supply lines are required.

Alternatively, each pressure zone can be formed by exactly one fluid cell.

The at least two fluid cells of the pressure zone can be arranged directly adjacent to each other. Preferably, a pressure zone with at least two fluid cells has a common supply to pressurize them together with fluid under pressure. This can simplify the design of the patient support.

In addition, at least two fluid-connected fluid cells of the pressure zone can be spaced apart from each other by at least one other fluid cell of another pressure zone. This enables a simpler design of the patient support, as several fluid cells spaced apart from each other can be subjected to the same pressure of a pressure zone without the need for separate components, in particular valves.

The fluid cell can be formed in one piece with the seating side of the base, in particular the entire base. Such a fluid cell is thus spatially fixed relative to the base.

Alternatively, the fluid cell may be completely enclosed by an inner wall of the fluid cell and have at least one opening for a supply fluid and/or connection to another fluid cell. Such a fluid cell may be connected to the base only by the feed. However, the fluid cells are preferably fixed in a shape relative to the base.

The patient support may include an immersion sensor. The immersion sensor is adapted to detect an immersion depth. For example, the immersion sensor may be adapted to detect whether a predefined immersion depth is exceeded.

The immersion depth can be detected as a curvature or an angle of an inner wall section. Alternatively, the immersion depth can be detected by a reduction in the distance between two inner wall sections or a partial section between the two inner wall sections, in particular relative to the maximum distance between the two inner wall sections or the partial section.

The inner end wall section is adjacent to the support areas so that the immersion depth can be detected in an operating position of the patient support essentially in a vertical direction.

The immersion sensor can comprise a contactless measuring device, in particular an optical or acoustic device. In particular, the immersion sensor can comprise an interferometer, a light barrier or a device for transit time measurement.

The immersion sensor can be arranged partially or completely in a pressure zone, in particular in a fluid cell.

The immersion sensor may be configured to detect contact between an inner wall section of at least one fluid cell and another element. The other element may preferably be an opposite inner wall section of the fluid cell, the base, and/or the immersion sensor.

The immersion sensor can be arranged in just one fluid cell or in several fluid cells, for example in a centric area of the patient support. This allows material costs to be saved and the patient support can be calibrated on the basis of the areas of the patient that sink in the most.

Additionally or alternatively, the immersion sensor can be arranged in a rear half of the patient support, preferably in a rear third of the patient support, and/or on a side area, so that detection of the sinking in of the ischial tuberosities and/or the breech area can be detected.

For example, the contact can be detected by the closing of an electrical circuit by at least two electrodes of the immersion sensor. One of the two electrodes can be arranged on one of inner wall sections of the fluid cell, the base or the immersion sensor. The other of the two electrodes can be arranged on the same or a different one of the inner wall section, the base or the immersion sensor.

Such an immersion sensor can therefore be particularly reliable and robust in the event of a defect, as it has a simple design and comprises few parts that can move in relation to each other. In addition, the immersion sensor is therefore independent of environmental influences that could affect a capacitive sensor, for example.

The immersion sensor can also be designed to determine the spatial position of the contact, for example by having a network of contacts.

The immersion sensor can alternatively or additionally comprise a resistive, inductive or capacitive sensor for detecting contact.

The immersion sensor can comprise two sections that are at least partially movable relative to each other. Alternatively or additionally, the immersion sensor can be at least partially deformable in one direction. Preferably, the immersion sensor is configured so that it triggers before the maximum immersion depth is reached.

This allows the immersion sensor to detect before the patient's endangered area is no longer sufficiently protected/relieved. The two sections of the immersion sensor can be moved perpendicular to each other.

Such an immersion sensor can be triggered by a predefined deformation of the immersion sensor.

The immersion sensor can be elastically deformable so that the immersion sensor returns to its original shape when an acting force of deformation is removed. An elastically deformable immersion sensor also enables a certain cushioning effect to be provided for the patient in the event of a defect.

The patient support can be a seat cushion and can be designed for use on a wheelchair. The seat cushion can have a maximum extension in the direction parallel to the support area of a maximum of 65 cm, preferably a maximum of 55 cm.

Patients sitting in a wheelchair place particular strain on the ischial region, especially the ischial tuberosities and the tailbone region. A seat cushion that is dimensioned for a wheelchair allows the patient to change positions optimally and can prevent damage to the overlying tissue, for example in the area of the ischial tuberosities.

The patient support can have a thickness perpendicular to the support area in a range from 4 cm to 20 cm, in particular a thickness in a range from 4 cm to 15 cm or from 4 cm to 10 cm.

Such dimensioning of the patient support allows the patient support to be easily transported. In addition, the pressure distribution of the patient can be optimized by maximizing the contact area of the patient support and thus protecting the vulnerable area by making the patient support wide enough to adapt to the contour of a patient.

The at least one pressure zone can have a different shape or surface area in plan view to another pressure zone.

In addition, one fluid cell of a pressure zone may have a different shape or surface area in plan view of the patient support compared to another fluid cell, in particular of the same pressure zone.

The different shape or surface area of the pressure zones/fluid cells makes it possible to better adapt the pressure zone/fluid cell to particularly vulnerable areas and to the contours of a patient.

One or more pressure zones/fluid cells can, for example, form a supporting structure around a pressure zone for holding the particularly vulnerable parts of a patient in order to relieve them.

The patient support can have an outlet valve and an inlet valve. The inlet valve can be fluid-connected to a pump. The outlet valve and inlet valve can be fluid-connected to at least one zone valve, preferably at least two zone valves, in particular preferably all zone valves.

Preferably, the patient support has exactly one outlet valve and/or preferably exactly one inlet valve. This has the advantage that the number of valves required for the patient support can be reduced. Only one additional zone valve per zone is sufficient, so that no separate outlet and inlet valves are required for the individual pressure zones.

In addition, the patient support can have a three-way valve so that the function of the outlet valve and inlet valve can be fulfilled by the three-way valve.

At least one valve, preferably all valves, of the group of the inlet valve, the outlet valve and the zone valve can be normally closed and preferably comprise a check valve assembly.

The backflow of fluid can be prevented in particular by a mechanical reset element, in particular comprising a spring and a seal, of the check valve assembly, so that no active energization of the valve in a closed state is required.

A de-energized closed valve is less susceptible to faults and also ensures that the fluid cannot escape completely from a pressure zone in the event of a power loss.

The pump and/or an exhaust silencer can have a noise level of less than 30 dB during operation, preferably less than 25 dB, particularly preferably less than 20 dB, for example measured at a maximum distance of 1 m from the patient support.

A low noise level of the pump and/or the exhaust silencer allows the patient support to be operated without disturbing the patient or people nearby, especially in the working environment, in everyday life or when sleeping.

The pump can be a diaphragm or ultrasonic pump. A diaphragm pump is particularly insensitive to continuous stress and contamination. This enables more durable patient support despite long-term operation.

The patient support can comprise an elastic cover. The cover is adaptable along a contour of the fluid cells when a patient's weight force is applied to the cover, without causing a significant force parallel to the support area.

Significant force in this context can typically be understood as a force parallel to the support area, the amount of which is ap-proximately less than 5 percent of the amount of the patient's weight force acting on the cover. Additionally or alternatively, the cover can also be designed such that a tensile stress in the cover of less than 30%, in particular 20%, of a contact pressure is caused by the weight force acting on the patient.

By applying a low force, parallel or perpendicular to the support area, the tensile stress of the cover can be minimized so that a so-called hammock effect can be avoided. The hammock effect is a support effect on the patient caused by the cover, which can distort the desired support effect of the fluid cells.

The coating can comprise a macroscopically stretchable structure. This macroscopically stretchable structure may comprise stitched or folded areas, protrusions and/or depressions. This macroscopically stretchable structure enables a reduced tensile stress of the cover when the force acting on the cover, in particular the weight force of a patient, is applied.

Such a cover makes it possible to provide the patient with the most point-elastic support possible through the fluid cells when lying down and avoid tension in the cover. The cover also distances the patient from the fluid cell material, which is often not very breathable, so that the overlying tissue is better ventilated.

The cover can also be air-permeable. This makes it easier to remove moisture from the support area and improves ventilation of the patient's overlying areas.

The coating may comprise or consist of a plastic, in particular a foam, preferably an open-cell/open-pore foam.

The patient support may comprise at least one humidity sensor and/or temperature sensor for measuring the humidity and/or temperature on the seat side of the base.

The control unit may be configured to adjust the air supply/extraction of spaces between the fluid cells on the basis of the measured humidity and/or temperature, in particular by means of at least one additional ventilation valve.

Alternatively or additionally, the patient support can include a visual or auditory notification element that can be activated by the control unit. The notification element can indicate the exceeding of a measured value or a defect by means of a light source or the sound of a loudspeaker. For example, if a zone valve is defective or if the temperature, humidity and/or pressure of a fluid cell/pressure zone is too low or too high, an alarm sound can indicate the defect.

Such moisture and/or temperature sensors can detect increased skin moisture/development of skin moisture at an early stage, which represents an additional risk factor for the development of tissue damage to the patient.

The patient support may include a ventilation device for the supply and/or removal of fluid on the seat side of the base. The ventilation device may be operable by the pump.

The ventilation device can be switchable and/or adjustable, preferably by the control unit, on the basis of a user input, a humidity measurement of the humidity sensor and/or a temperature measurement of the temperature sensor.

The ventilation device can comprise, for example, a fan, a pump and a ventilation system with fluid inlets and outlets. Fluid can thus be removed from or supplied to the seating area by the ventilation device.

The fluid supply/fluid discharge can be connected, for example via a ventilation valve, to the space between the fluid cells, preferably to a plurality of spaces. In addition, the fluid supply/fluid discharge may have a plurality of fluid supply channels that lead to different spaces between the fluid cells in the seating area.

The ventilation device may further comprise a dehumidification unit configured to reduce the moisture content of supplied fluid by a method known to the person skilled in the art, in particular based on heat exchange.

In this context, it would also be conceivable that the ventilation device is configured to dehumidify an interior of the fluid cells, the valves and/or the supply, for example to compensate for condensation caused by pressure differences.

The control unit or an additional computer arrangement of the patient support can be designed to detect temporal pressure changes in at least one pressure zone by at least one pressure sensor and/or immersion sensor and to assign them to an activity pattern. For example, the control unit can measure pressure values at regular time intervals. Alternatively, a frequency of pressure changes can be measured, as can occur during sport, for example.

The pressure changes over time can be recorded, for example, by measuring repeatedly within predefined time intervals or by detecting the time of the pressure change depending on when predetermined pressure changes are exceeded.

In this context, the activity pattern is typically a temporal sequence of pressure changes in the pressure zones caused by movements of the patient that have a defined periodicity and/or intensity.

For example, a low-intensity pressure change that rarely occurs over a longer period of time, such as when the patient is asleep, can be assigned to a specific activity pattern.

The activity patterns can include an activity pattern for high activity such as exercising, moderate activity such as eating or reading, and/or low activity such as resting or sleeping. The activity pattern can also be assigned to an individual movement pattern of the patient.

An activity pattern can be assigned using a predefined algorithm, in particular by exceeding predefined threshold values. The threshold values can, for example, represent a predetermined frequency, periodicity and/or intensity of pressure changes within a predefined period of time. The algorithm can also be adapted to the patient's weight.

Alternatively, the assignment of an activity pattern can be done by classification using machine learning.

The algorithm or machine classification for assigning an activity pattern can be patient-specific, in particular based on the patient's anatomy.

By assigning a specific activity pattern, the control unit can optimize control in such a way that tissue damage can be prevented for each patient activity.

The patient support can be operated in a first and a second operating mode. It is also conceivable to provide further operating modes. The control unit can be designed to control the pressurization of the pressure zones so that the first operating mode has a first pressure ratio between at least two pressure zones. In the second operating mode, a second pressure ratio different from the first can be set between the two pressure zones.

The control unit can be configured to change the operating mode based on the detection of a certain activity pattern.

The different operating modes allow individualized adjustment of the pressurization of the patient support to the patient's needs.

The control unit can be designed in such a way that in the first or second operating mode, a temporally repeating pressurization and pressure relief of at least one pressure zone, in particular groups of pressure zones, takes place.

Varying the pressurization over time can prevent a patient from being exposed to one-sided stress over a long period of time and thus minimize tissue damage.

Alternatively or additionally, the control unit can be designed in such a way that an activity pattern of a patient is detected in the first or second operating mode and the pressurization of the pressure zones is adapted to this activity pattern.

Alternatively or additionally, the control unit can be designed in such a way that in the first or second operating mode, pressurization of the pressure zones takes place for stable positioning of the patient.

The patient support is therefore suitable for a wide range of applications with different patient activity patterns.

Alternatively or additionally, the control unit can be designed in such a way that in the first or second operating mode, pressurization of the fluid cells, preferably all fluid cells, up to a maximum pressure takes place.

Pressurization up to the maximum pressure can make it easier to transport the patient from or to the patient support.

The seat side of the base can have no fluid cell in one area. Preferably, an edge of the base or an area adjacent to the edge is designed without fluid cells. The control unit and/or the pump can be arranged in this area of the base.

Certain areas of the patient support often do not accommodate particularly vulnerable parts of the patient, so these areas do not necessarily need to be supported by fluid cells. By positioning the control unit/pump in one of these areas, a more compact patient support can be provided.

The control unit and/or pump can be arranged partially or completely in a common plane with the plurality of fluid cells. Thus, the thickness of the patient support can be reduced perpendicular to the base.

In particular, the control unit and/or pump can be arranged in an area of the patient support which, in the operating position, is intended to accommodate a part of the patient that is not particularly at risk of tissue damage.

The parts of the patient that are not particularly at risk are typically parts that are at a distance from the patient's center of gravity and/or have an even weight distribution. For a seated patient, an area that is not particularly at risk is the lower thigh, for example.

The patient support can have a pressure port. At least one additional device can be pressurized by the pump through the pressure port if the additional device is connected to the patient support via the pressure port.

The pressure port can be closed in the standard state and can only be opened by connecting the additional device. The additional device can be an extension of the patient support, which can be connected to the patient support and in particular comprises one or more fluid cells that form at least one pressure zone.

The patient support can therefore be easily extended and adapted for a wide range of other applications.

A system may comprise a patient support as described above and such an additional device. The system can thus, for example, in addition to supporting the patient's buttocks by the patient support, support a patient's back by the additional device. The additional device could also be another patient support.

An overpressure valve can be assigned to at least one fluid cell and/or pressure zone, preferably at least one fluid cell per pressure zone.

An overpressure valve can avoid load peaks in the fluid cells or pressure zones and thus prevent damage to the patient support.

The fluid cells may comprise or consist of a. Preferred plastics are silicones, vulcanized rubber, polyurethane, polychloroprene, poly(organo)siloxane, polyisoprene, polyethylene, polypropylene, polystyrene, and/or polyester. The fluid cells preferably comprise or consist of a film material, in particular a plastic film. The plastic film preferably comprises or consists of one of the aforementioned plastics.

These plastics are gas-tight and sufficiently elastically deformable.

The base can also consist of or comprise the plastic materials described. A base made of such plastic material enables a flexible base of the patient support so that it can adapt optimally to a support area, in particular a seat surface, such as that of a wheelchair.

The patient support may include a wireless communication interface for sending status data of the patient support and/or receiving user input data, in particular for selecting the operating mode for operating the patient support.

Such a wireless communication interface enables uncomplicated monitoring of patient positioning status data and more convenient operation by user input.

The wireless communication interface can communicate with a user's terminal device, such as a smartphone, tablet or laptop, preferably in a frequency band between 2.402 GHZ and 2.480 GHz.

The patient support can comprise a data memory and a computing unit. The computing unit can be used to calculate at least one target pressure value of at least one pressure zone based on at least one measured value of the immersion sensor and/or the pressure sensor. The measured values and/or the target pressure value can be stored in the data memory. The control unit can be used to set a pressure in at least one pressure zone based on the target pressure value.

The pressure within the fluid cells or pressure zones may be adjustable for a specific patient. In particular, the pressure can be adjustable on the basis of the pressure values previously measured by the sensors, in particular pressure values on the basis of a previous calibration. In this way, the patient support can provide individual support that is optimized for the respective patient.

A wheelchair may include a patient support as described above.

The invention further relates to a method for automated calibration of a patient support. The method is preferably used for calibrating a patient support as previously described. The method comprises a step of placing a patient on the patient support so that a weight force is applied to the patient support.

The pressure of at least one pressure zone is varied. The pressure at which an immersion sensor detects a sit-through is determined. Sitting through can be detected by an immersion sensor described above. In particular, sit-through can mean that an inner wall section of at least one fluid cell of the pressure zone touches another element, preferably an opposite inner wall section of the fluid cell, a base and/or the immersion sensor.

The determined pressure is saved.

When varying the pressure of at least one pressure zone, the immersion depth can be measured as a function of the pressure. By measuring the immersion depth, the calibration can be further optimized.

This procedure can be repeated for another pressure zone, a plurality of pressure zones, in particular two to five, or all pressure zones.

Using such a method, a patient support can be automatically adapted to the individual needs of a patient. Depending on the patient's anatomy, in particular weight, height and weight distribution across the fluid cells/pressure zones, a different pressure is measured at which the immersion sensor detects a pressure zone.

The operating modes can be adapted to the patient on the basis of these values. For example, a minimum pressure in the fluid cells can ensure that the patient does not sag during operation.

A target pressure value in at least one pressure zone can be determined based on the determined pressure of a pressure sensor at which the immersion sensor detects. Determining a target pressure value for a pressure zone enables patient-specific adaptation of individual pressure zones.

The immersion sensor can be arranged in a pressure zone. The pressure of the pressure zone and at least one adjacent pressure zone can be adjusted on the basis of the determined pressure of the pressure sensor at which the immersion sensor detects.

Thus, not only the pressure change of a fluid cell/pressure zone can be taken into account, but also the interaction of a pressure change of a fluid cell/pressure zone on nearby, in particular neighboring, pressure zones can be taken into account. In-creasing or decreasing the pressure of at least one neighboring pressure zone makes it possible to relieve or load a specific pressure zone in particular.

The invention is described below with reference to certain embodiments and figures showing:

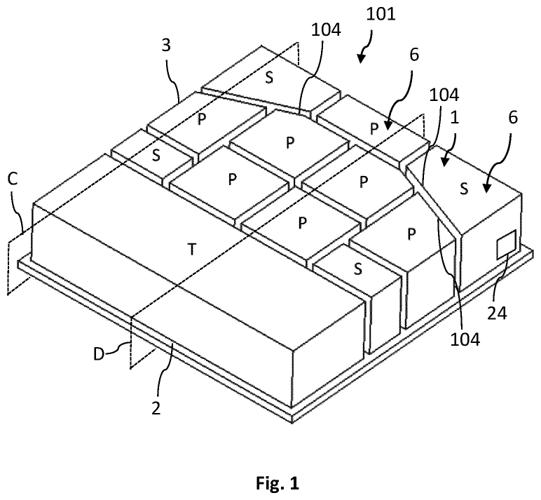

FIG. 1: An oblique side view of a first embodiment of a patient support;

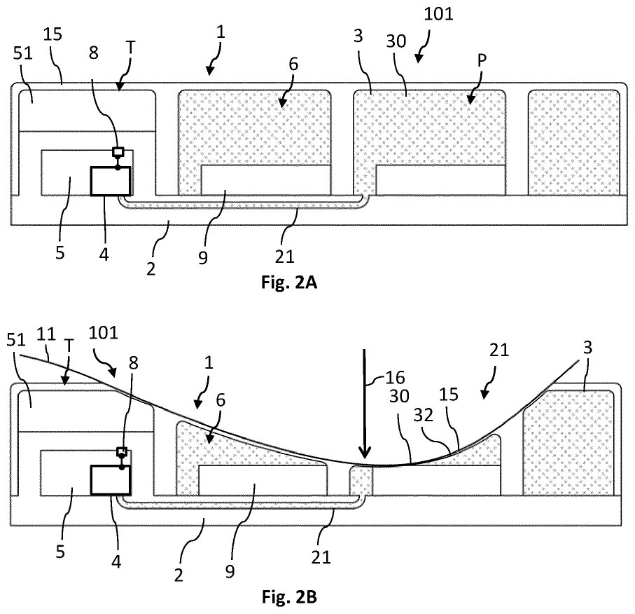

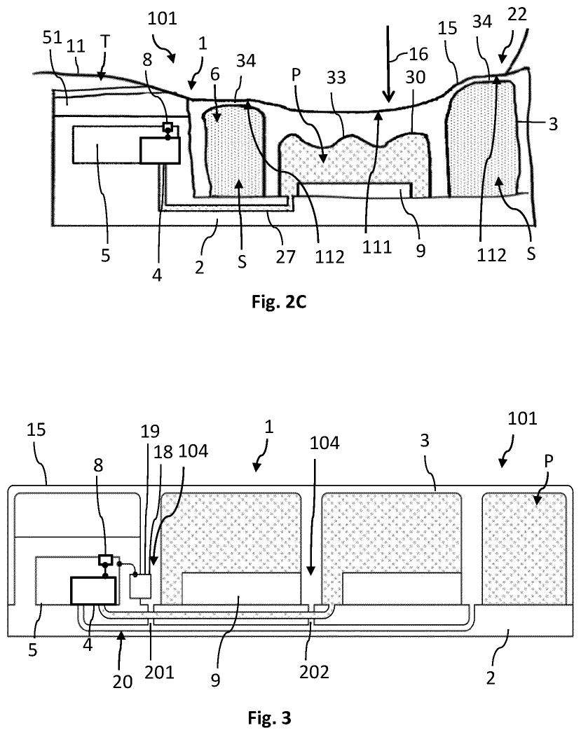

FIGS. 2A and 2B: a cross-section D of the patient support of FIG. 1 without the application of force and under the application of force

FIG. 2C: a cross-section C of the patient support of FIG. 1 under the action of force;

FIG. 3: a cross-section of a second embodiment of a patient support with a ventilation device;

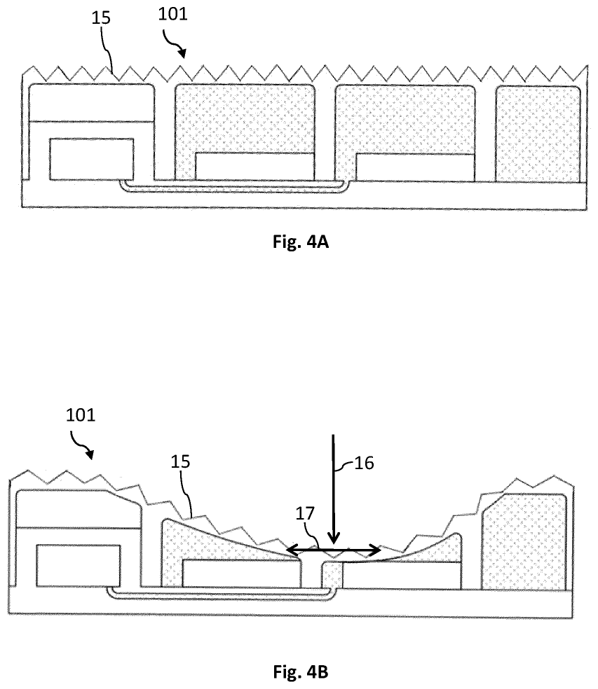

FIGS. 4A and 4B: a cross-section of a schematic representation of a patient support with a cover with and without an acting force parallel to the cover;

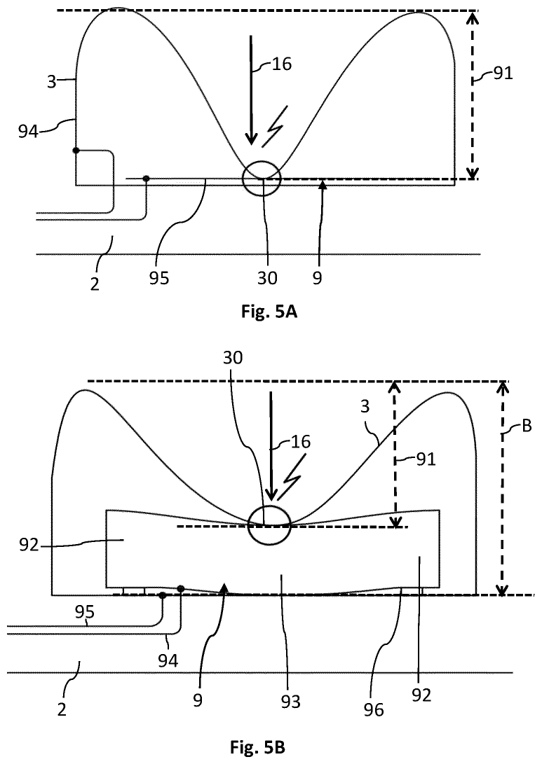

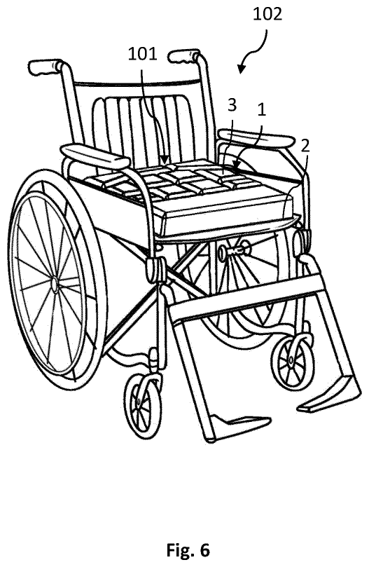

FIGS. 5A and 5B: a first and second embodiment of a fluid cell with an immersion sensor in cross-section;

FIG. 6: An oblique side view of a wheelchair with the embodiment of the patient support shown in FIG. 1;

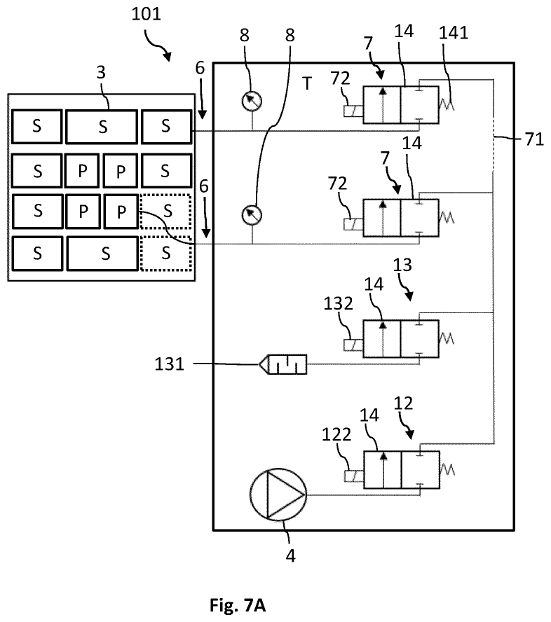

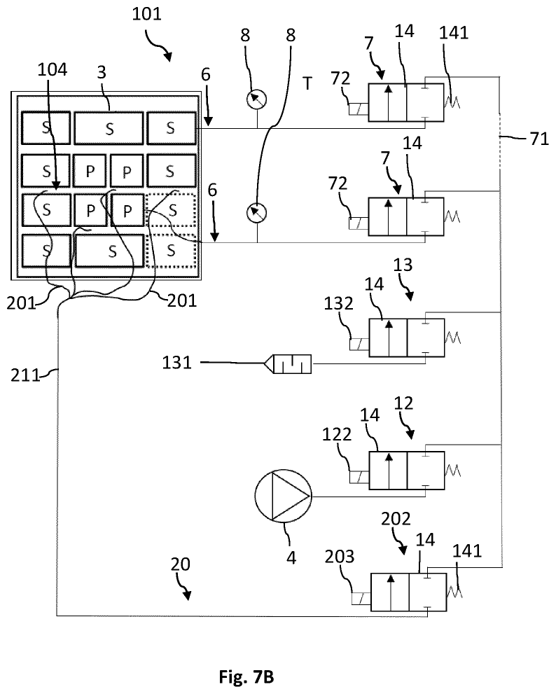

FIG. 7A: a schematic representation of the valve control of a patient support according to FIG. 2A to FIG. 2C;

FIG. 7B: a schematic representation of the valve control of a patient support according to FIG. 3;

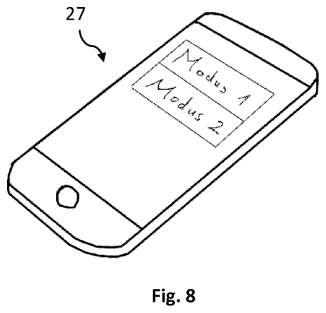

FIG. 8: an oblique side view of a user terminal for communication with a patient support;

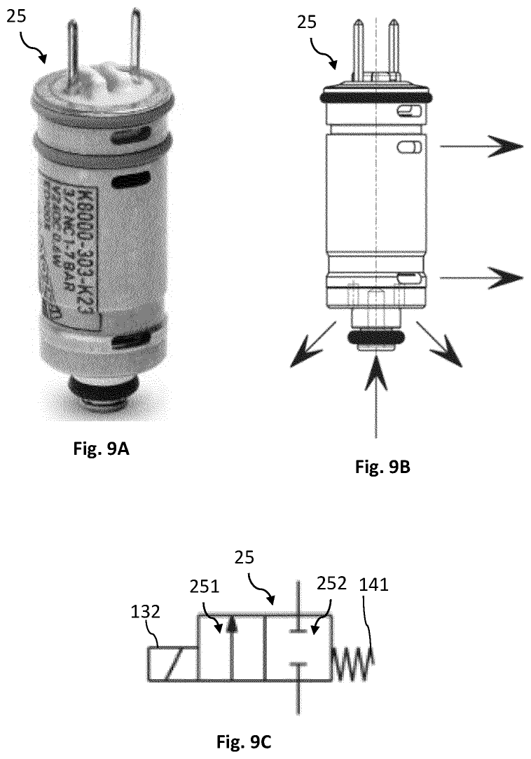

FIGS. 9A to 9C: a first embodiment of a valve;

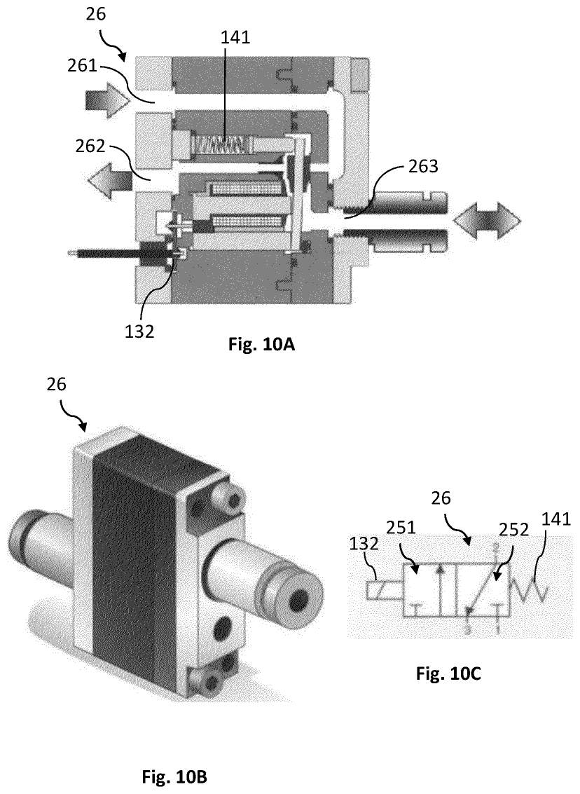

FIGS. 10A to 10 C: a second embodiment of a valve;

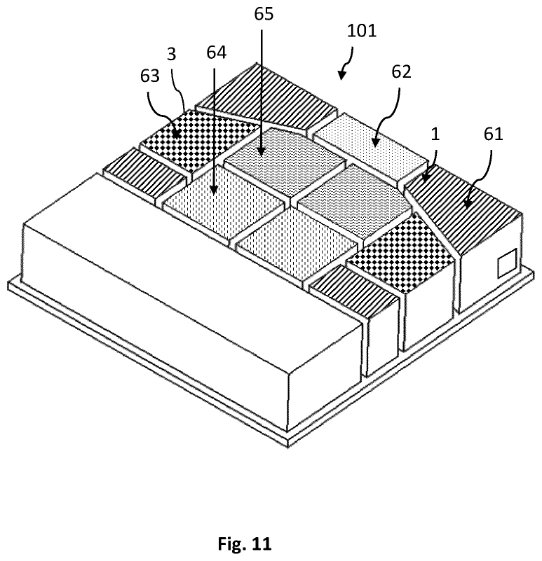

FIG. 11: An oblique side view of a third embodiment of a patient support.

FIG. 1 shows an oblique side view of a first embodiment of the patient support 101 in the shape of a seat cushion. This embodiment of the patient support 101 is intended for use in a wheelchair and has a seat area 1, which is arranged on a base 2 of the patient support 101. The base 2 is made of a flexible material, such as polypropylene, polyethylene, or rubber, so that the base 2 can adapt to a contour of a support, for example a wheelchair. In addition, the patient support 101 can thus be transported more easily.

The patient support 101 has a control area T in which a control unit is arranged. The control area T is arranged at one edge of the patient support 101 so that a patient with the lower thighs can be arranged on it, as this part of the body is not particularly at risk of pressure sores. The control area T only has a flexible cushion for cushioning the patient and no fluid cell 3. Alternatively, however, it is also conceivable to equip the control area with one or more fluid cells or pressure zones. The patient is adequately supported by the flexible cushion from the electronic components and a pump within the control area T.

In addition, the patient support 101 has a plurality of fluid cells 3 made of polyurethane film, which form the seat area 1 with the control area T and are materially bonded to the base 2. The fluid cells 3 in FIG. 1 each form a pressure zone 6. FIG. 1 further shows seven pressure zones 6 for primary support P and four pressure zones 6 for secondary support S. However, it would also be conceivable that the area of primary and secondary support P, S comprises pressure zones formed by several fluid cells 3 (see FIG. 11). The fluid cells 3 are shaped differently so that the patient can be better supported. The geometric shape and size of the fluid cells 3 is adapted to the patient's support requirements. The ischial tuberosities and the patient's tailbone area are particularly relieved by the primary support P, as this area represents a part of the body particularly at risk of decubitus ulcers. Some side surfaces 104 of the fluid cells 3 of the primary support P and secondary support S are, in plan view, partially shaped at an angle to a rectangular edge area of the base 2 in order to frame the region at risk. In this way, the support of the patient can be optimized.

A pressure port 24 is provided in the rear area of the patient support 101, which is intended for connecting and applying compressed air to other devices. For example, a patient's back can be supported by a device that can be pressurized with compressed air, in particular another patient support that can be connected to the patient support 101. The schematic cross-section D shown in FIG. 1 through the centric fluid cells 3 of the patient support 101 is shown in FIG. 2A and FIG. 2B. The cross-section C schematically shown in FIG. 1 in an edge region of the patient support 101 is shown in FIG. 2C.

FIG. 2A shows a cross-section of the patient support 101 of FIG. 1 without acting force. An elastic cover 15 is arranged on the seat area 1 and is adaptable to a contour 32 of fluid cells 3 (see FIGS. 4A and 4B). The patient support 101 also has an electronic control unit 5, which is arranged in the control area T. A flexible foam cushion 51 is arranged above the control unit 5 and covers the entire control area T. The control area T has no fluid cell 3. A pump 4 is arranged inside the control unit 5, with which the fluid cells 3 can be pressurized with compressed air, in that the pump 4 draws in ambient air and feeds it to the fluid cells 3 through supply lines 27. The pump 4 is a piezoelectric ultrasonic pump, which is known to the person skilled in the art, and causes a noise level of less than 20 dB at a distance of one meter during operation. In addition, the control unit 5 has a pressure gauge 8 for measuring the pressure of the pressure zones 6. In FIG. 2A to FIG. 2C, the pressure gauge 8 is connected to the control unit 5 and arranged at the end of the feed 27 so that it can be fluidly connected to the pressure zones 6 in order to measure the pressure. Thus, in FIGS. 2A to 2C, the pressure gauge 8 can be connected to different pressure zones 6 by valves which are controllable by the control unit 5 to measure the pressure of the pressure zones 6 (not shown in FIGS. 2A to 2C). The zone valves and inlet and outlet valves are all arranged in the area of the control device 5, so that no additional lines need to run through the base 2 to control the valves. However, in an alternative embodiment, each pressure zone has its own pressure gauge (see FIG. 7).

In addition, the two central fluid cells 3 of the pressure zones 6 for primary support P each have an immersion sensor 9, which is connected to the control unit 5 and detects when a predefined immersion depth of the fluid cell 3 is exceeded (see FIGS. 5A and 5B). The immersion sensor 9 in FIG. 2A and FIG. 2B is arranged in the lower area of the fluid cell 3 adjacent to the base 2, so that exceeding the immersion depth of the patient's ischium or tailbone area, which sink in particularly deeply, can be detected. This predefined immersion depth is achieved in FIG. 2B by an inner wall section 30 of the fluid cell contacting the immersion sensor 9 so that an electrical contact is closed (see FIG. 5A and FIG. 5B). The control unit 5 is configured to control the pump 4 on the basis of the measured pressure values of the pressure gauge 8 and/or immersion depth of the immersion sensor 9. The individual pressure zones 6 can be separately supplied with compressed air by the pump 4 through separate supply lines 27 with separate zone valves 7 (see FIG. 7).

For better visibility, only a supply line 27 for compressed air to a pressure zone 6 in the primary supporting area P, which extends within a base 2, is shown in FIGS. 2A to 2C. However, the three fluid cells 3 in FIG. 2B for the primary support P can each be pressurized separately by a supply line 27. The supply lines 27 each have a zone valve in the area of the control device 5, so that they can be selectively pressurized. The primary area P is shown dotted for better visibility in FIG. 2A and FIG. 2B. The fluid cells 3 are each connected to the base 2. Alternatively, the fluid cells 3, which form a common pressure zone 6, can be fluidly connected to each other by at least one line (see FIG. 11).

FIG. 2A shows the patient support 101 without an acting force, so that no deformation of the seat area 1 takes place.

FIG. 2B shows the patient support 101 with the acting weight force 16 of a patient 11 in the first operating mode 21. In this exemplary first operating mode 21 in FIG. 2B, the control unit 5 is configured to support the patient as evenly as possible by the fluid cells 3 while avoiding sagging of the patient 11. As a rule, the patient 11 should not be supported on a hard surface, except for calibration, for example, but should be supported by the fluid cells. In particular, the patient should not normally sink into the surface of the immersion sensor 9. However, the control unit 5 in the first operating mode 21 is configured to ensure the highest possible immersion depth during proper operation without triggering the immersion sensor 9, so that a support surface that supports the patient 11 can be maximized.

For this purpose, the control unit 5 can be calibrated by reducing the pressure within at least one fluid cell 3. The immersion sensors 9 can thus detect at which fluid cell 3 and preferably at which pressure value of the manometer 8 the patient 11 exceeds the predefined immersion depth. Exceeding the predefined immersion depth, or the pressure value, is shown in FIG. 2B by contact between the upper inner wall section 30 of the fluid cell 3, which is deflected by the weight force 16, and the immersion sensor 9. When the predefined immersion depth is exceeded, an electrical contact of an electrical circuit of the immersion sensor 9 is closed and the immersion sensor 9 triggers.

Alternatively, detection can take place by interrupting the electrical contact

In addition, calibration with an initially non-pressurized fluid cell 3/pressure zone 6 would also be conceivable. For such a calibration, the pressure of fluid cell 3 is increased instead of reduced. Meanwhile, the remaining fluid cells 3 can be pressurized with a reference pressure, in particular a minimum or maximum pressure. The immersion sensor 9 accordingly detects the pressure at which the inner wall section 30 no longer contacts the immersion sensor 9 due to the weight force 16, for example due to a loss or establishment of electrical contact of the immersion sensor 9. The circuit can therefore be closed or open when the inner wall section 30 contacts the immersion sensor 9 and, conversely, open or closed when the inner wall section 30 is removed from the immersion sensor.

The control unit 5 can be calibrated patient-specifically on the basis of this detected value or a plurality of such values for different fluid cells 3. For this purpose, the control unit 5 has a computing unit with an internal electronic data memory. In particular, optimal target pressure values for the fluid cells 3 can be determined by the computing unit of the patient support 101 and stored on the data memory. In this way, the surgical modes 21, 22 can be adapted to the individual needs of the patient 11. The patient-specific values and operation modes 21, 22 are stored in the data memory for the patient so that the same patient 11 does not have to be recalibrated.

In addition, the preceding calibration can ensure that an upper inner wall section 30 of the fluid cell 3 cannot be deflected to the base 2 in the operating mode 21, 22. The various pressure zones 6 can be pressurized with compressed air at a different ratio of pressure to each other depending on the operating mode 21, 22.

FIG. 2C shows a cross-section of the patient support 101 with the acting weight force 16 of the patient in an edge region of the patient support 101 in a second operating mode 22. In this exemplary second operating mode 22, the control unit 5 is configured to particularly relieve a critical ischial or breech region 111 of the patient 11. For this purpose, the fluid cells 34 of a secondary support S around the fluid cell 33 that receives the critical ischial area 111 of the patient 11 are pressurized with compressed air to a greater extent than the fluid cell 33 that receives the critical ischial area 111. The area for secondary support S is shown in FIG. 2C as a narrow dotted line for better visibility and the centric area for primary support P is shown as a coarse dotted line. By varying the support of the neighboring areas 112 of the critical area 111, damage to the tissue of the patient 11, in particular the formation of pressure sores, can be avoided. In FIG. 2C, only a single fluid cell 33, which forms a pressure zone 6, is shown as an example to accommodate the critical area 111. However, a pressure zone 6 comprising several fluid cells 3 is also conceivable, which is relieved by one or more neighboring pressure zones 6 (see FIG. 11).

The patient support 101 also has many different such operation modes 21, 22. The control unit 5 is also configured to automatically select and adjust the operation modes 21, 22 based on the movement of the patient 11.

The movement of the patient 11 is assigned to an activity pattern by measuring temporal pressure changes in a pressure zone 6 by the manometer 8 and/or the immersion sensor 9. Based on the activity pattern, an operating mode 21, 22 is set or adjusted.

In addition, the control unit 5 is configured to switch more frequently between the operation modes 21, 22 depending on the assigned activity pattern, so that one-sided loads are avoided. In the case of an activity pattern with little movement of the patient 11, for example when the patient 11 is resting or sleeping, the operating mode 21, 22 is changed particularly often by the control unit 5. The change between operation modes 21, 22 is repeated periodically by the control unit 5.

In the case of an activity pattern with a lot of movement of the patient 11, for example during sports, a different operating mode 21, 22 is automatically set by the control unit 5. The operating mode 21, 22 is also configured by the control unit 5 in such a way that the positioning of the patient is optimized when there is a lot of movement by applying significantly more compressed air to the supporting and less vulnerable area S of the patient support 101 than to a centric primary support area P.

The control unit 5 also has a wireless communication interface for sending and receiving data. The user can preferably easily connect a smartphone to the communication interface via an app. The user can thus view the status data of the patient support 101, in particular status data or patient data stored on a data memory of the patient support 101. In addition, the user can send an input to the communication interface, for example to change the operating mode 21, 22. The computing unit of the control unit 5 is also configured to make the patient-specific adjustments to the operation modes 21, 22 on the basis of the user input. Based on the user input, certain preferences for control-ling the operating modes 21, 22, such as a time period after which the operating mode 21, 22 is changed, can be adjusted. In addition, the calibration of the patient support can be performed by a user input, stored and automatically assigned to a specific user.

In addition, the user can set an operating mode 21, 22 for transporting the patient 11, for example. In these operating modes 21, 22, all fluid cells 3 are fully pressurized with compressed air so that the patient 11 can be easily lifted and/or slid off the patient support 101.

FIG. 3 shows a cross-section of a second embodiment of the patient support 101 with a ventilation device 20. The ventilation device 20 is connected to a pump 4, so that the ventilation device 20 can be operated with the pump 4. The ventilation device 20 has a plurality of accesses 201, 202 to the spaces 104 between the fluid cells 3. The accesses 201, 202 of the ventilation device 20 extend through a base 2 of the patient support 101. The air circulation from a seat side of the base 2 of the patient support 101 is thus improved either by air supply or air removal, driven by the pump 4. A cover 15 over the fluid cells 3 is designed to be permeable to air, so that parts of a patient lying on it can be better ventilated. This has the advantage that the formation of moisture through perspiration of the overlying areas can be reduced. The risk of softening of the skin due to moisture accumulation and the associated reduction in skin resilience can therefore be reduced.

FIG. 3 also shows a control unit 5, which is connected to a humidity sensor 18 and a temperature sensor 19. The humidity and temperature sensors 18, 19 are partially arranged in the intermediate space 104 so that the temperature and humidity in a seating area 1 of the patient support 101 can be detected. Alternatively or additionally, one or more humidity and temperature sensors 18, 19 may be arranged between the fluid cells 3 of the particularly vulnerable area for primary support P. The primary support area is shown dotted in FIG. 3 for better visibility. In addition, as in FIG. 2A to FIG. 2C, the control unit 5 is connected to a pressure gauge 8 and an immersion sensor 9.

The control unit 5 has a computer unit which operates the ventilation device 20 on the basis of the measured values of the humidity and/or temperature of the humidity and temperature sensors 18, 19. The computing unit is also configured to control the patient support 101 on the basis of a pressure determined by the pressure gauge 8 and/or a detected immersion depth of the immersion sensor 9, analogous to FIGS. 2A to 2C.

FIGS. 4A and 4B show a cross-section of a schematic representation of a first embodiment of the patient support 101 according to FIG. 2A and FIG. 2B with a macroscopically stretchable cover 15. Such a cover can also be used for the second embodiment in FIG. 3 or third embodiment of the patient support 101 in FIG. 11.

No weight force acts on the cover 15 in FIG. 4A, so that a flexible structure, which is schematically represented by a jagged pattern, is not deformed. The cover 15 is made of an open-cell polyurethane foam, for example, so that it can be deformed under low force while still allowing air to flow to the patient's overlying tissue.

The flexible structure can be deformed parallel to a transfer surface even with a small amount of force. The flexible structure is formed by seams, folds and/or easily deformable material.

In FIG. 4B, a weight force 16 acts on the cover 15. The flexible structure 15 is deformed by the acting weight force 16 in a direction 17 parallel to the cover 15, but without generating strong tensile stresses in the cover 15. This prevents a “hammock effect”, which could cause localized damage to the patient's overlying tissue due to tension in the cover. The weight of the patient is thus essentially absorbed point-elastically by the fluid cells of the patient support located under the cover 15, whereby the cover 15 adapts to the contour of the fluid cells due to the weight force.

FIGS. 5A and 5B show two embodiments of a fluid cell 3 with an immersion sensor 9 in cross-section. In FIG. 5A and FIG. 5B, a current source is connected to a first conductor 94 and/or second conductor 95. The immersion sensor 9 detects when these two conductors 94, 95 contact each other and thus close a circuit.

Alternatively, it could be detected if a circuit is interrupted or if a detected resistance value of a circuit changes.

The embodiment of the fluid cell 3 in FIG. 5A shows that at a maximum immersion depth 91, an upper inner wall section 30 contacts the first conductor 94 of the fluid cell 3 with the second conductor 95. The second conductor 95 of the immersion sensor 9 is arranged on a side facing a base 2 at the bottom of the fluid cell 3, so that an acting weight force 16 triggers the immersion sensor 9.

The embodiment of the fluid cell 3 in FIG. 5B shows that the immersion sensor 9 has two different sections 92, 93, which are movable relative to each other. Only the outer sections 92 of the immersion sensor 9 are held by supports 96. The middle section 93, on the other hand, can be deflected downwards by the weight 16 of a patient. The middle section 93 of the immersion sensor 9 thus has a supporting effect, so that a patient is supported even if the fluid cell 3 is defective. In addition, the high degree of elasticity on contact prevents a potentially dam-aging pressure effect on the ischial or coccyx areas to be protected. The maximum immersion depth 91 of the fluid cell is significantly less than the entire width B of the fluid cell 3.

The first conductor 94 in FIG. 5B is electrically connected to the middle section 93 and the second conductor 95 is electrically connected to a bottom of the fluid cell 3, which runs parallel to the base 2. By deflecting the middle section 93 and contacting the bottom, the immersion sensor 9 in FIG. 5B can thus be triggered.

FIG. 6 shows an oblique side view of a wheelchair 102 with an embodiment of the patient support 101 according to FIG. 1 with a plurality of fluid cells 3 on a base 2, which form a seating area 1. The patient support 101 can be used as a detachable seat cushion of the wheelchair 102 or can be rigidly connected to the wheelchair 102.

FIG. 7A shows a schematic representation of the valve control of the embodiment of the patient support 101 according to FIG. 2A to FIG. 2C, with the difference that the patient support 101 has a separate pressure gauge 8 for each pressure zone 6 for measuring the pressure in the pressure zone 6. The pressure zones 6 are each formed by a fluid cell 3. Alternatively, however, a pressure zone 6 can be formed by several fluid cells 3 that are fluid-connected to one another, so that the fluid cells 3 can each be pressurized with compressed air at a uniform pressure (see FIG. 11).

An ultrasonic pump 4 with an inlet valve 12 and an outlet valve 13 are arranged in a control area T of the patient support 101. The inlet valve 12 is connected to the diaphragm pump 4, so that a supply 71 to a plurality of zone valves 7 can be pressurized with compressed air. The outlet valve 13 is also connected to the feed 71 to the zone valves 7. Thus, one zone valve 7 is required per pressure zone 6 of an area for primary and secondary support P, S. The inlet valve 12 and outlet valve 13 can thus be used for pressurizing with compressed air or discharging compressed air from all pressure zones 6. This means that fewer valves 7, 12, 13 are required per pressure zone 6. The inlet valve 12 and the outlet valve 13 are also optimized for a low noise level of less than 20 dB at a distance of one meter during operation. The outlet valve 13 also has a silencer 131 for this purpose.

The plurality of zone valves 7 is shown by only two different pressure zones 6 for better clarity. However, the dotted area of the feed 71 indicates that the patient support 101 has further pressure zones 6, each formed by a fluid cell 3. Alternatively, several fluid cells could also form a pressure zone. All valves 12, 13, 7 also have an electrical actuation 72, 132, 122, so that the valves 12, 13, 7 opened/closed electronically individually by a control unit.

In addition, the valves 12, 13, 7 comprise a check valve assembly 14 as a safety measure, which is closed by a return spring 141 in a de-energized state. The check valve assembly 14 can be opened by energizing it via the control unit. In this way, compressed air can be supplied through the inlet valve 12 or air can be discharged through the outlet valve 13.

FIG. 7B shows a schematic representation of the valve control of the embodiment of the patient support 101 according to FIG. 3. The valve control in FIG. 7B is similar to FIG. 7A, but in contrast to FIG. 7A also has a ventilation device 20. For a further description of the preceding features, please refer to FIG. 7A.

The ventilation device 20 is formed by a fluid supply 211, which is connected to the piezoelectric ultrasonic pump 4 via a ventilation valve 202, alternatively also a plurality of additional ventilation valves 202. The fluid supply 211 is divided, on a side facing the fluid cells, into a plurality of fluid supply channels 201 which extend to a plurality of intermediate spaces 104 of the fluid cells 3. Thus, uniform ventilation of the interstices 104 of the patient support 101 can be achieved so that skin moisture can be reduced and moisture accumulation can be avoided.

The aeration valve 202 in FIG. 7B is shaped substantially analogous to the inlet valves 12, the outlet valve 13, and the zone valves 7 and also has a check valve assembly 14 with a return spring 141 and an electric actuator 203. The electrical actuation 203 can be controlled by a control unit to open the aeration valve 202 so that fluid, in particular air, is fed through the fluid supply 211 from the piezoelectric ultrasonic pump 4 to the interstitial spaces 104. Thus, the ultrasonic pump 4 can be used both to apply fluid to the fluid cells 3 and to provide ventilation through the ventilation device 20.

FIG. 8 shows a schematic representation of a user terminal device in the shape of a smartphone 27. The smartphone can be wirelessly connected to a communication interface of a patient support according to FIG. 2A to FIG. 3 via an app, so that it can send user input and read out status data of the patient support. In addition, a user can set a preferred operating mode of the patient support by user input (see FIGS. 2A to 2C).

FIGS. 9A to 9C show a first embodiment of a valve 25 which can be used as an inlet, outlet, and/or zone valve 12, 13, 7 (see FIG. 7). The valve 25 has an open state 251 which allows a flow of compressed air through the valve. The valve 25 also has a blocked state 252, in which no compressed air can flow through the valve 25. As a safety measure, the valve 25 is in a de-energized state in the blocking state 252. The blocking state 252 of the valve can be transferred to the open state 251 by actuating an electronic actuator 132 against a return force of a return spring 141

FIGS. 10A to 10C show a second embodiment of a valve 26 which can be used as an inlet, outlet and/or zone valve 12, 13, 7 (see FIG. 7). The valve 26 is a three-way valve, so that a first valve access 261 is provided for an introduction of compressed air and a second valve access 262 is provided for a discharge of compressed air from a pressure zone. A third valve access 263, which can be fluidly connected to the first and second valve access 261, 262, can, on the other hand, supply compressed air from a pump to the pressure zone or discharge it from the pressure zone to an outlet. The valve 26 can be closed by a spring 141 in a de-energized state, i.e. it can assume a blocking state 252. To discharge compressed air, the spring 141 can also be deflected by an electrical actuation 132, so that the valve 26 assumes the open state 251 and compressed air can be discharged through the second valve access 262.

FIG. 11 shows a third embodiment of a patient support 101 in the shape of a seat cushion. This third embodiment of the patient support 101 differs from the patient support 101 of FIG. 1 and FIG. 3 in that several fluid cells 3 form different pressure zones 61, 62, 63, 64, 65 in a seat area 1. The respective pressure zones are each marked with a different pattern for better recognizability.

The fluid cells 3 of a pressure zone 61, 62, 63, 64, 65 are each fluid-connected to one another by lines, so that they can be jointly supplied with compressed air at a uniform pressure. In addition, the respective pressure zones 61, 62, 63, 64, 65 each have only one common supply line from a pump of the patient support 101 to one of the fluid cells 3 of the pressure zone 61, 62, 63, 64, 65. One of the pressure zones 61, 62, 63, 64, 65 each has only one zone valve.

The fluid cells 3 of two pressure zones 61, 63 are spaced apart in plan view by fluid cells 3 of other pressure zones 62, 64, 65. This arrangement makes it possible, for example, to take into account the often partially mirror-symmetrical seating surface of a patient without having to calibrate pressure zones separately from each other.

1.-28. (canceled)

29. A patient support comprising
a support area with a base,
a plurality of fluid cells arranged on a seating side of the base,
a pump,
and a control unit,
wherein one or more fluid cells each define a pressure zone and each pressure zone is connected to the pump by at least one zone valve, wherein each zone valve can be actuated by the control unit,
so that a pressure in each pressure zone can be set by the control unit,
wherein the patient support comprises at least one pressure sensor for measuring the pressure in at least one pressure zone.

30. The patient support according to claim 29, wherein the patient support comprises an immersion sensor, wherein the immersion sensor is adapted to detect a contact between an inner wall section of at least one fluid cell with at least one of an opposite inner wall section of the fluid cell, the base, and the immersion sensor.

31. The patient support according to claim 29, wherein the patient support is a seat cushion and is designed for use on a wheelchair.

32. The patient support according to claim 29, wherein the patient support comprises an outlet valve and an inlet valve, wherein the inlet valve is fluid-connected to the pump and the outlet valve and the inlet valve are fluid-connected to at least one of one zone valve, at least two zone valves, and all zone valves.

33. The patient support according to claim 29, wherein at least one of the group of the inlet valve, the outlet valve, and the zone valve is normally closed.

34. The patient support according to claim 29, wherein at least one of: the pump is a diaphragm or ultrasonic pump and at least one of the pump and an exhaust silencer has a noise level of less than 30 dB at a distance of 1 m from the patient support during operation.

35. The patient support according to claim 29, comprising an elastic cover, wherein the cover, when a force of a patient acts on the cover, is adaptable along a contour of the fluid cells without causing a significant force parallel to the support area.

36. The patient support according to claim 29, wherein the control unit or an additional computer arrangement of the patient support is designed to detect temporal pressure changes in at least one pressure zone by at least one pressure sensor or the immersion sensor and to assign them to an activity pattern.

37. The patient support according to claim 29, wherein the patient support can be operated in at least one first operating mode and one second operating mode, wherein the control unit is designed to control the pressurization of the pressure zones, so that the first operating mode has a first pressure ratio between at least two pressure zones and the second operating mode has a second pressure ratio, different from the first, between the two pressure zones.

38. The patient support according to claim 37, wherein the control unit is designed such that in the first or second operating mode at least one of:
(i) a temporally repeating pressurization and pressure relief of at least one pressure zone takes place,
(ii) an activity pattern of a patient is detected and the pressurization of the pressure zones is adapted to this activity pattern,
(iii) the pressurization of the pressure zones is carried out for stable positioning of the patient, and
(iv) the fluid cells are pressurized up to a maximum pressure.

39. The patient support according to claim 29, wherein the base has no fluid cell on the seat side in a region and at least one of the control unit and the pump is arranged in this region of the base.

40. The patient support according to claim 29, wherein the patient support has a pressure port, with which at least one additional device can be pressurized by the pump when the additional device is connected to the patient support via the pressure port.

41. The patient support according to claim 29, wherein at least one fluid cell is assigned an overpressure valve.

42. The patient support according to claim 29, wherein the patient support comprises a wireless communication interface for at least one of transmitting status data of the patient support and receiving user input data.

43. The patient support according to claim 29, wherein the patient support comprises a data memory and a computing unit,
wherein at least one target pressure value of at least one pressure zone can be calculated with the computing unit based on at least one measured value of at least one of the immersion sensor and pressure sensor,
wherein at least one of the measured values and the target pressure value can be stored in the data memory and
a pressure in at least one pressure zone can be set by the control unit based on the target pressure value.

44. A wheelchair comprising a patient support according to claim 29.

45. A method for automated calibration of a patient support comprising the following steps:
placing a patient on the patient support so that a weight force is exerted on the patient support;
varying a pressure of at least one pressure zone and determining at which pressure an immersion sensor detects a sit-through;
save the determined pressure.

46. The method according to claim 45, wherein when varying the pressure of at least one pressure zone, the immersion depth is measured as a function of the pressure.

47. The method according to claim 45, wherein a target pressure value in at least one pressure zone is determined based on the determined pressure of the pressure sensor at which the immersion sensor detects.

48. The method according to claim 45, wherein the immersion sensor is arranged in a pressure zone, wherein the pressure of the pressure zone and at least one adjacent pressure zone are adjusted on the basis of the determined pressure of the pressure sensor at which the immersion sensor detects.
